(19) United States
(12) Patent Application Publication
(10) Pub. No.: US 2010/0330058 A1
(43) Pub. Date: Dec. 30, 2010

(54) CHEWABLE DRUG

(75) Inventor: Judy Davis, San Clemente, CA (US)

Correspondence Address: THE ECLIPSE GROUP LLP, 6345 Balboa Blvd., Suite 325, Encino, CA, 91316, US

(73) Assignee: Hero Nutritionals, LLC, San Clemente, CA (US)

(21) Appl. No.: 12/547,355

(22) Filed: Aug. 25, 2009

Related U.S. Application Data
(60) Provisional application No. 61/220,111, filed on Jun. 24, 2009.

(51) Int. Cl. A61K 38/43; A61K 47/36; A61K 47/42; A61K 36/00

(52) U.S. Cl. 424/94.1; 514/774; 514/778; 424/725

(57) ABSTRACT

A chewable composition for delivering pharmaceutical compounds. The chewable composition includes a drug delivery vehicle and an active pharmaceutical ingredient. The delivery vehicle may include an organic or non-organic gummy candy. The active ingredient may include an over-the-counter drug or a prescription drug to provide a desired effect on the user. In addition to the active pharmaceutical ingredient, the chewable composition may also include any combination of nutraceuticals, vitamins, minerals, antioxidants, soluble and insoluble fiber, herbs, plants, amino acids, and digestive enzymes.

### RELATED APPLICATIONS

This application claims priority from U.S. Provisional Application No. 61/220,111, filed on Jun. 24, 2009, titled CHEWABLE DRUG, which application is incorporated in its entirety by reference in this application.

### BACKGROUND OF THE INVENTION

1. Field of the Invention

This invention relates to a chewable drug, and more particularly, to a chewable delivery system for the delivery of pharmaceutical compounds, and a method for manufacturing the same.

2. Related Art

Over-the-counter (OTC) and prescription drugs play a vital role in America's health care system. While both are regulated by the U.S. Food and Drug Administration (FDA), OTC drug products are available to consumers without a prescription. There are more than 80 therapeutic categories of OTC drugs, ranging from acne drugs products to weight control products, and more than 100,000 OTC drug products are currently sold in this country. OTCs are critical to our country's health care system because they provide easy access to certain drugs that can be used safely without the help of a health care practitioner.

Despite their necessity, ingestion of OTC and prescription drugs can be, for many, an unpleasant experience. Most individuals take several drugs orally each day. Drugs that are taken orally generally come in the form of a liquid, a tablet, a pill, or a capsule. In many cases, this is not an enjoyable experience because the pharmaceuticals are either difficult to ingest due to size or texture, or are not palatable.

Attempts to overcome the bad taste characteristics of pharmaceutical compounds due to acidity, bitterness, burning in the back of the throat, or odor, have been made. Such formulations attempt to overcome the unpleasant taste characteristics of these drugs by coatings, capsules, flavoring agents, or combinations of these features. But these prior art delivery agents are not ideal because when they are manufactured, the chemical composition and/or potency of the drug is altered and/or diluted.

Recently, chewable supplements have been manufactured and sold in the form a gummy candy supplement. Now a selection of vitamins is being manufactured and sold in a chewable gummy form, including both children's and adult supplements. The introduction of gummy supplements into the marketplace has been particularly helpful in getting children to take daily vitamin supplements. For adults that do not like swallowing pills, gummy supplements have also provided a non-pill alternative for adults to get their daily vitamin requirements.

Although gummy candy was first introduced in 1920 as “gummy bears,” it was not until very recently that gummy candy was first utilized, by Hero Nutritionals, LLC, San Clemente, Calif., as a delivery system for dietary supplements. Traditional gummy candy is made from a gelatin base—which is similar to the base found in soft caramels, marshmallows, foam-filled wafers, licorice, wine gums, pastilles, chocolate coated mallows and a host of other sweets—and a blend of corn starch, corn syrup, sugar, color, and flavor. Gelatin gives the candy its elasticity, the desired chewy consistency, and a longer shelf life.

To date, no one has utilized gummy candy as a delivery system for pharmaceuticals. A need exists in the art for a drug delivery system that enables drugs to be easily and quickly digested by users of all ages, and may be manufactured without compromising the potency of the drug.

### SUMMARY

A chewable composition for delivering pharmaceutical compounds is provided. The chewable composition generally includes a binding agent, a sweetener, and a pharmaceutical compound.

According to one implementation, the chewable composition includes a drug delivery system for delivering pharmaceutical compounds to a user's body. The drug delivery system includes a drug delivery vehicle in the form of a gummy candy, and a pharmaceutical compound as a primary active ingredient of the gummy candy. In particular, the delivery vehicle may include a non-organic or an organic gummy candy. The gummy candy may include sweeteners, a binding agent, and natural and/or artificial flavors, colors, and preservatives. For example, in one implementation, the gummy candy may include glucose syrup, natural cane juice, gelatin, citric acid, lactic acid, natural colors, natural flavors, fractionated coconut oil, and carnauba wax.

The primary active ingredient may include an OTC or a prescription drug to provide a desired effect on the user. In addition to the primary active ingredient, the drug delivery system may also include nutraceuticals (i.e., extracts of food purported to have a medicinal effect on human health) such as botanical and herbal extracts and antioxidants, or any combination of food supplements such as vitamins, minerals, soluble and insoluble fiber, herbs, plants, amino acids, and digestive enzymes.

According to another implementation, a method of preparing a chewable drug is provided. The method includes mixing a binding agent with water to form a gelling compound, adding a desired dosage of a pharmaceutical compound to the gelling compound, and adding a sweetener to the gelling compound. After the sweetener is added to the gelling compound, the compound is cooked to form a cooked candy, and then flavoring may be added to the cooked candy. After the flavoring is added, the cooked candy is molded and cured until a chewable drug is formed.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 shows a flow diagram of an example of a method of manufacturing a chewable drug of the present invention.

FIG. 2 shows a flow diagram of an example of a method for incorporating a pharmaceutical compound into a delivery vehicle of a drug delivery system of the present invention.

### DETAILED DESCRIPTION

The present invention relates to a chewable drug, in particular, a delivery system that is chewy or gummy-like and designed to enhance the delivery of pharmaceutical compounds. The delivery system includes a primary active ingredient to provide a desired effect, and a chewable delivery vehicle to contain the active ingredient for delivery.

The primary active ingredient of the present invention may include a pharmaceutical compound. In one implementation, the pharmaceutical compound may include an OTC drug to treat symptoms of common illnesses. Such OTC drugs may include Benadryl®, Sudafed®, Claritin®, Maalox®, Mylanta®, Insulin, Tums®, Pepcid® AC, Monistat®, Ex-Lax®, Imodium® A.D., Robitussin®, Chloraseptic®, Thera-flu®, Alka-Seltzer, Motrin®, Dramamine®, and the like, in liquid or powder form. In another implementation, the pharmaceutical compound may include a prescription drug. Such prescription drugs such may include Lipitor®, Singulair®, Lexapro, Plavix®, Morphine, Hydrocodone (Vicodin®), Demerol®, Codeine, Diazepam (Valium®), Penicillin, Prevacid®, Allegra-D®, Celebrex®, Crestor®, Cialis®, Valtrex®, Ambien CR®, Viagra®, Flomax®, Prozac®, and the like, in liquid or powder form. As for the dosage, pharmaceutical compounds are generally expressed in terms of grams or milligrams, but may also be expressed in active units, or international unit (IU). As used herein, a “pharmaceutical compound” or “drug” shall include, but is not limited to, any drug, hormone, peptide, nucleotide, antibody, or other chemical or biological substances used in the treatment or prevention of disease or illness, or substances which affect the structure or function of the body.

In addition to an active pharmaceutical ingredient, the delivery system may also include any combination of vitamins, minerals, antioxidants, soluble and insoluble fiber, herbs, plants, amino acids, digestive enzymes, or any other supplements digested to promote the heath and well-being of a person. Inclusion of any of these dietary supplements will depend in part on their compatibility with the pharmaceutical compound.

The primary active ingredient is delivered in a delivery vehicle that is palatable and easy to swallow. In one implementation, the delivery vehicle may be a gummy candy to facilitate swallowing. The delivery vehicle may include a binder, sweeteners, and flavoring. The delivery vehicle may further include coloring and preservatives.

### Manufacturing of Delivery System

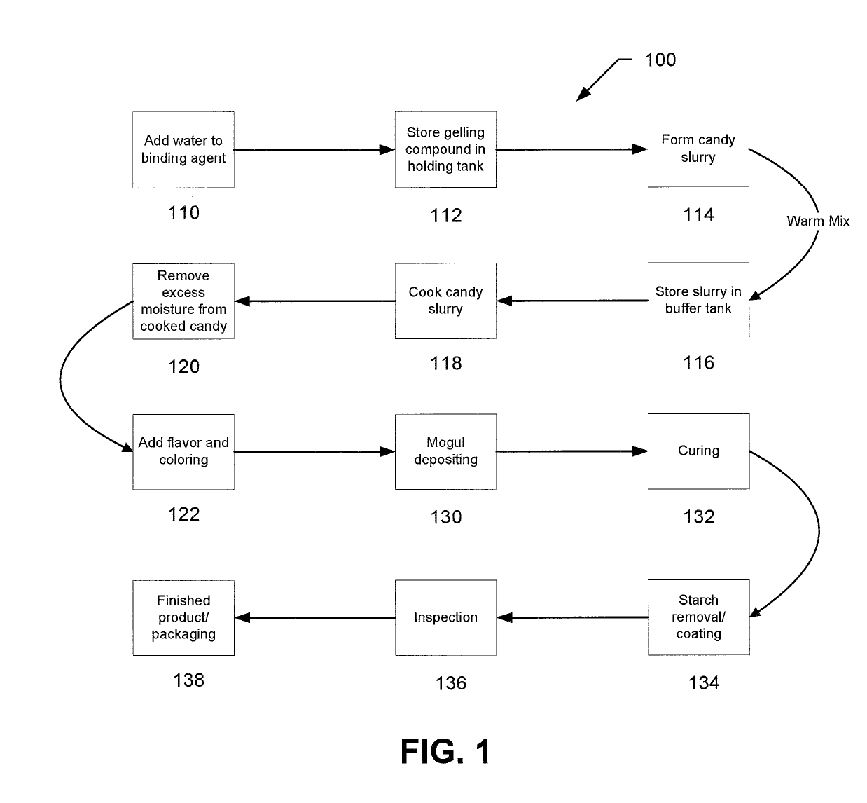

Turning now to FIG. 1, a method 100 for manufacturing a delivery system of the present invention is disclosed. In general, the method of manufacturing involves three main phases: (i) mixing and storing; (ii) batching and cooking; and (iii) depositing and curing.

The first phase of mixing and storing begins with step 110, where water and a binding agent are mixed in a mixing tank to form a gelling compound. In one implementation, the mixing tank may include a 1,000 gallon stainless steel planetary mixer, a scrape surface mixer, a holding tank with an agitator, or any other suitable mixer. During production, water and the binding agent are continuously mixed in the mixing tank and the gelling compound is continuously turned in the tank by an agitator to keep the binding agent suspended in water (i.e., to prevent the binding agent from settling on the bottom of the mixing tank). In one implementation, approximately 6,000 lbs to 8,000 lbs of gelling compound may be produced in 8 hours.

The gelling compound may include cold, warm, or hot water. However, warm or hot water may be used to reduce the hydration time (i.e., the time it takes the water to hydrate the binding agent) of the gelling compound. For example, about 250 lbs of gelatin mixed with about 250 lbs of warm water may reach a homogenous mixture in about 10 minutes. The hydration rate of the gelling compound may also vary according to the speed of the agitator.

The binding agent may include a pectin gel, gelatin, food starch, or any combination thereof. Depending on the binding agent used, the gelling compound may include, for example only, one of the following formulations:

TABLE A
GELLING COMPOUND FORMULA

| | | Water (% |
|---|---|---|
| Binding Agent | Binding Agent (% by weight) | by weight) |
| gelatin | 50% | 50% |
| pectin | 2%-3% | 97%-98% |
| starch | 7%-10% | 90%-93% |
| pectin/starch | 8%-10% (1%-2% pectin/7%-8% starch) | 90%-92% |

In one implementation, a buffer, such as sodium bisulfate or sodium citrate, may be mixed into the gelling compound to regulate the pH of the mixture. In one implementation, the gelling compound may contain approximately 0.01 to 0.03% buffer by weight, or any other suitable amount. The pH of the mixing tank may be adjusted to a range from about 3.2 to about 4.5 to provide adequate gelation and to ensure that the gelling compound does not become unstable (or acidic) during mixing.

At step 112, the gelling compound may be filtered through a fine mesh, to remove particulates in the slurry, and stored in a holding tank. In one implementation, about 140 lbs to 190 lbs of gelling compound may be delivered from the mixing tank to the holding tank every 5 to 10 minutes. The filter may be a 0.034 inch stainless steel basket strainer and the holding tank may be a 1,500 gallon stainless steel tank. In some implementations, the holding tank may include a moderate agitator (e.g., mixing blades) to mix the compound and prevent the binding agent from settling on the bottom of the holding tank during storage.

From the holding tank, approximately 125 lbs to 185 lbs of gelling compound may be delivered to a mixing vessel at step 114, every 5 to 10 minutes, for example. In one implementation, the mixing vessel may be a 5,000 gallon stainless steel planetary mixer. In other implementations, the mixing vessel may be a scrape surface mixer, a holding tank with an agitator, or any other type of suitable mixer.

In the mixing vessel, water, additives, supplements, and an active ingredient may be added to the gelling compound to form a sugar slurry. In one implementation, the additives may include sodium citrate, sweeteners such as sugar (also referred to herein as sucrose or natural cane juice) and/or syrup (e.g., corn, glucose, rice, tapioca), and corn starch, in liquid and/or powdered form. In one implementation, the supplements may include vitamins, minerals, herbs, plants, amino acids, enzymes or any other supplements digested to promote the heath and well-being of a person.

The supplements may include, but not be limited to, any of the following:

Vitamin B1 (Thiamine)

Vitamin B2 (Riboflavin)

Vitamin B3 (Niacinamide)

Vitamin B5 (Pantothenic Acid)

Vitamin B6 (Pyridoxine HCL)

Vitamin B12

Biotin

Folic Acid

Vitamin C (Ascorbic Acid/Activated C)

Calcium

Carotine

Chromium

Choline

Copper

Magnesium

Zinc

Protein

Pomegranate

Inositol

Vitamin D (Cholecalciferol)

Vitamin E (Acetate)

Gingseng

Iron

Vitamin K (Phytonadione)

St. John's Wort

The above list of raw materials are not exhaustive, but are provided for illustrative purposes only. The length of a list of all available supplements that may be utilized in the chewable supplement of the invention is too lengthy to provide.

In one implementation, the sugar slurry may contain approximately 70% to 85% sweetener by weight, while the remaining approximately 15% to 30% of the slurry (by weight) may contain the gelling compound and additives. More particularly, the slurry may contain approximately 19% water, 2% sodium citrate, 30% natural cane juice, 45% corn syrup, 3% supplements, and 1% primary active ingredient by weight. In most implementations, the candy slurry may reach a homogeneous mixture in about 5 to 10 minutes.

The ingredients described above and their compositions are provided by way of example only. Without departing from the spirit and scope of the present invention, the ingredients and the composition of the sugar slurry may vary based on the type of formulation desired. For example, corn starch may be added to the sugar slurry in an implementation where pectin is added to the gelling compound, to stabilize the product; or, to reduce production cost, the sweetener may include a combination of natural cane juice (sugar) and syrup. In addition to reducing production cost, syrup may also be used to temper the resulting candy.

Prior to production, the sugar and syrup additives may be stored in bulk tanks. In one implementation, the syrup may be stored in a holding tank at a temperature of approximately 75° F. In the holding tank, the syrup may be irradiated by ultraviolet light to remove any contaminants in the syrup. The syrup may include high fructose corn syrup (e.g., HFCS-42, HFCS-55, or HFCS-62), glucose syrup, rice syrup, tapioca syrup, or any other suitable liquid sweetener or combination thereof. During production, the syrup may be administered to the mixing vessel manually or by automation.

Similarly, prior to production, sugar may be stored in a holding tank. During production, sugar may be fed through an automated feed system that filters the sugar to remove any sediments, weighs the sugar, and delivers a desired quantity of sugar to the mixing vessel. In other implementations, sugar may be added to the mixing vessel manually.

Turning back to FIG. 1, from the mixing vessel, the sugar slurry is processed through a magnetic device, which removes particulates from the slurry, and stored in a storage buffer tank at step 116. In one implementation, the magnetic device may be a finger magnet or any other suitable magnetic device, and the storage tank may be a 5,000 gallon stainless steel industrial holding tank. In other implementations, the holding tank may include a moderate agitator to suspend the active ingredients in the sugar slurry. Prior to reaching the storage buffer tank, the sugar slurry may be heated through a series of heat exchangers to a temperature of approximately 150° F. to 180° F.

In one implementation, the storage buffer tank may receive the candy slurry from the mixing vessel at a mass flow rate of approximately 15 lbs/s to 20 lbs/s, and maintain the slurry at a temperature of approximately 150° F. to 200° F. Simultaneously, the warm candy slurry may be continuously fed from the storage buffer tank to a static cooker at mass flow rate of approximately 10 lbs/s to 15 lbs/s, by way of example only.

In the next phase of batching and cooking, at step 118 the candy slurry mix is received by the static cooker and cooked at a temperature of approximately 220° F. to 260° F. for approximately 30 sec. to 60 sec., until the slurry is gelatinized (i.e., dehydrated). In one implementation, the static cooker may be a 2,500 gallon high pressure steam jacketed kettle, a vacuum pressure cooker, or any other suitable cooker. In the static cooker, moisture is evaporated out of the candy slurry as the slurry is boiled to a temperature of approximately 250° F. After about a minute of boiling, the slurry may consist of about a 65 to 75 brix solution (i.e., the slurry may consist of approximately 65 grams to 75 grams of sugar per 100 grams of solution).

After the candy slurry is cooked, the cooked candy is then delivered to a vacuum chamber at step 120. In one implementation, the vacuum chamber may be a 50 psi industrial vacuum chamber or any other suitable enclosure. In another implementation, the pressure cooker may also include a vacuum apparatus.

In the vacuum chamber, moisture is drawn from the cooked candy by suction pressure. In one implementation, the vacuum chamber may draw out approximately 2% to 5% water by weight. At this juncture, the cooked candy may have a brix of approximately 67 to 80, and a pH of approximately 2.8 to 4.0, for example.

From the vacuum chamber, the cooked candy is filtered into a trough, commonly known as a dosier. In one implementation, the filer may be a 0.034 inch basket strainer. At this point in the manufacturing process, the cooked candy mainly consists of a clear sugar gelatin. To obtain a desired color and taste, coloring and flavoring may now be added to the cooked candy.

At step 122, the cooked candy may be passed through the dosier. In the dosier, water, flavoring, coloring, and food acid may be added to the cooked candy to enhance the candy's taste. For example, flavoring such as artificial and/or natural flavoring (e.g., fruit concentrate) may be added to the cooked candy to give the candy a desired flavor. To balance the flavor, food acid may be added to the cooked candy. Such food acids may include citric acid, ascorbic acid, malic acid, lactic acid, adipic acid, fumaric acid, tartaric acid, or any other suitable food acid or combinations thereof. In one implementation, the flavoring, coloring, and acid may be continuously added to (e.g., dripped on) the cooked candy as the candy moves through the dosier to the mogul machine.

The amount of flavoring, coloring, and acid administered to the cooked candy may vary according the volume of cooked candy passing through the dosier and the desired candy formulation. For example, in one implementation, approximately 1% to 2% flavoring by weight and approximately 0.01% to 0.03% acid by weight may be added to the cooked candy composition. However, the amount of acid and flavoring added to the cooked candy formulation must be balanced to insure that the cooked candy will taste good. So, depending on the formulation, more flavoring and less acid may need to be added to the cooked candy for bitter formulations. In some instances, only food acid instead of flavoring may be added the cooked candy.

In addition to food acid, coloring and titanium dioxide may be added to the cooked candy formulation in the dosier. Coloring may be added to give the candy a desired color or colors. Coloring may include natural coloring such as black carrot, annatto, tumeric, and purple berry concentrate, or artificial coloring such as yellow 5, red 3, and blue 1.

Titanium dioxide may be added to the candy to provide sheen. Titanium dioxide may also stabilize the cooked candy formulation so the coloring does not bleed when it is handled, packaged, or stored.

In the final phase of depositing and curing, after the cooked candy is passed through the dosier, the candy may be sent to a starch molding machine at step 130. In one implementation, the starch molding machine may include a mogul machine (simply referred to as a “mogul”). A mogul is a starch molding machine that deposits the cooked candy, or gummi stock, onto starch covered mold boards (“mogul boards”) that allow the cooked candy to firm and take on the shape of the mold, to produce a series of shaped gummy candies. The mogul boards are usually covered with starch to prevent the gummi stock from sticking to the mogul boards.

In some cases, the starch used to coat the mogul boards may include recirculated starch prepared from re-used starch that is sifted and dried in a starch dryer, and then cooled in a starch cooler. The cooled starch is sifted again and placed in the mogul where it is recirculated through the same process. The recirculated starch may then be sprayed evenly on the mogul board. The cooked candy may then be deposited onto mogul boards coated with the recirculated starch.

After the cooked candy is placed on the mogul boards, the cooked candy is placed in a temperature and humidity controlled curing room, where the candy sits and cools (i.e., is cured) for approximately 24 hours to 48 hours (step 132). Proper curing time is necessary to ensure sugar, oil, or wax coating and ease of packaging without breakage and proper yield. In one implementation, the candy may be cured in a curing room with approximately 15% to 25% humidity.

After curing, the gummy candies, firmed and having proper texture, may be removed from the mogul boards and dumped into a tumbler machine at step 134. In one implementation, the tumbler may be a 2,000 gallon rotating drum. In the tumbler, the gummies may be tumbled together to remove any starch remaining on the gummy candies. Once the starch is removed, the gummies may become sticky, so the gummies may be polished or coated with oil to prevent the cooked candies from sticking together. Depending on the desired finished product or preferences, the gummies may be polished with fractionated coconut oil, linsic oil, bees wax, carnauba wax, or any other suitable food grade oil. In other implementations, the gummies may be sanded with sugar in a sugar drum.

After the gummies are coated, they are placed on a cooling belt (e.g., a conveyor belt) and transported to an inspection station at step 136. At step 136, the gummy candies are placed on an inspector belt where the candy is inspected for food safety and proper organoleptic effects. For example, on the inspector belt the gummy candies may be passed by a detector or x-ray to insure that no particulate or other foreign material has been deposited into the candy during the depositing stage.

Moving on to step 138, once the candy passes inspection, the finished gummy product is packaged for distribution.

The disclosure above only describes one implementation of a method of manufacturing a delivery system of the present invention. Other methods and implementations may be used to manufacture delivery systems in accordance with the present invention.

### Addition of Pharmaceutical Compound

Pharmaceutical compounds may be incorporated into a delivery system of the present invention by one of three methods: (i) as a liquid or solid prior to cooking the gummy composition; (ii) by encapsulation; or (iii) in liquid or extract form after the gummy composition has been cooked. The manner in which a pharmaceutical is incorporated into the delivery system depends on the heat sensitivity and chemical composition of the drug.

For example, under the first method, a drug may be added to the gelling compound at step 114 (FIG. 1), during the mixing and storing phase. In one implementation, the drug may be poured into the mixing vessel in solid, powdered or liquid form.

Because many pharmaceutical compounds are destroyed or degraded when exposed to heat, this method may not be effective for heat-sensitive drugs. For instance, in the mixing phase of the gummy manufacturing process, the gelling compound may be heated to a temperature of 185° F. Thus, the chemical structure of a drug incorporated into the delivery system under this method must be able to withstand temperatures in excess of 200° F.

For heat-sensitive drugs, such as probiotics, the second method of encapsulation may be applied. Under this second method, the drug may be encapsulated before it is added to the gelling compound at step 114 (FIG. 1). This method may be most effective for drugs, in solid or powdered form, that are moderately resistant to heat.

Prior to encapsulation, the drug may be pulverized to within a discrete particle size ranging from approximately 10 microns to 300 microns; the smaller the particle size, the more effective the encapsulation. Because the drug is encapsulated, the drug release and absorption capabilities of the delivery system may be controlled depending on the effectiveness of the encapsulation. For example, encapsulation may prevent early release of the drug to the user's system.

In one implementation, a solvent system containing a filming agent may be mixed with the drug particles and blended at slow speed in a planetary mixer. The solvent may be water or ethanol and the filming agent may be ethylcellulose, gelatin, a water-soluble plasticizer (e.g., glycerin, xylitol, or glucose), or any other suitable composition. The filming agent solution may be slowly added to the drug particles so that enough individual particles will adhere together to form larger granules having a size of approximately 300 to 500 microns. The degree of encapsulation may vary depending upon the number of layers of filming agent solution applied. In one implementation, the film coating may have a thickness of about 1 micron or less. There exist various standard pharmaceutical coating techniques that are suitable for use with this invention, depending on the filming agent, type of active ingredient that is to be coated, and the drug release objective, such as immediate release vs. sustained release.

Under the third method, heat sensitive drugs may be added to the cooked candy at step 122, during the flavoring and coloring phase. In one implementation, a drug in liquid or extract form may be added to the cooked candy in the dosier with the coloring and/or flavoring. While in other implementations, the drug may be added in solid or powdered form, drugs in the form of liquid or extracts are preferred at this stage of the manufacturing process because liquids and extracts are better absorbed by the cooked candy.

The amount of flavoring added to the cooked candy will vary depending upon the desired flavor and amount of pharmaceuticals added to the gelling compound. Some pharmaceuticals will require differing amounts of flavor, sweetener, color, and citric acid to create a desirable tasting chewable drug. For example, to mask the flavor of a particular drug, a flavoring agent such as strawberry flavor or cherry flavor may be added to the mixture. The additional flavor would be adjusted based upon the drug. For extra bitter drugs, a flavor masking flavor compound from flavor houses may also be utilized.

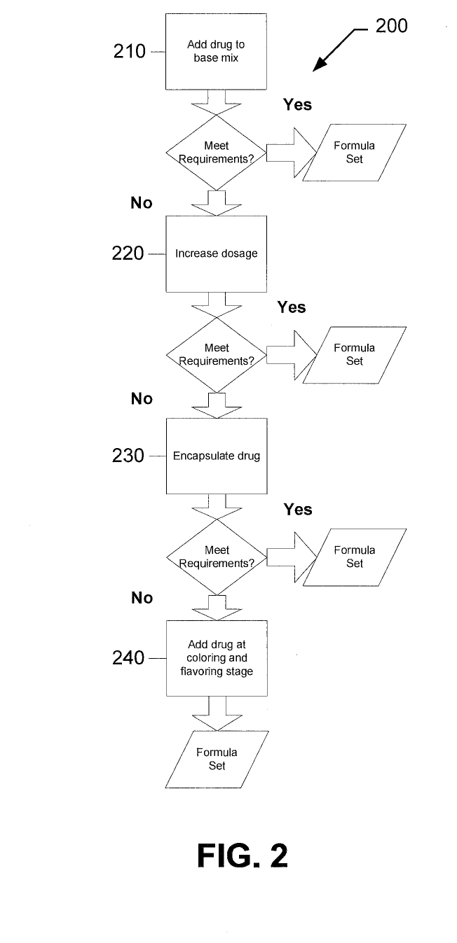

Turning now to FIG. 2, one implementation of a method 200 of incorporating a pharmaceutical into the delivery system of the invention is described. According to this method, the first step (step 210) is to prepare a test batch of gummy candies adding the drug to the gelling compound in the mixing vessel, at step 114 (FIG. 1) of the manufacturing process. After the gummy candy is cooked, cooled and cured, the candies may be inspected and tested at step 136 (FIG. 1) to validate that the drug composition of the candies meet the desired label requirements (i.e., meet the dosage printed on the product label). If the drug composition is validated, then the chemical formulation of the finished gummy product is set and the gummy candies may be mass produced and packaged using the first method of incorporation described above.

If the drug composition is not validated (i.e., the drug composition breaks down because the drug is heat sensitive), a second test batch may be produced and tested. This time, the dosage of the drug added to the gelling compound at step 114 (FIG. 1) may be increased to compensate for the drugs broken down during the cooking phase (step 220). For example, if 100 mg of aspirin is added to the gelling compound in the mixing weigh vessel to produce a 75 mg drug, but only 50 mg of aspirin is measured in the finished product, then 150 mg of aspirin may be added to the gelling compound in the mixing weigh vessel during the second production to compensate for the 25 mg of aspirin dissipated during the manufacturing process.

Once tested, if the drug composition is validated, then the chemical formulation of the finished gummy product is set and the gummy candies may be mass produced and packaged using the first method of incorporation described above. However, if second batch does not meet the label requirements, the drug may need to be encapsulated or added at a different stage of the manufacturing process.

If encapsulation is required, then a third test batch of gummy candies may be produced (step 230). In this step, the encapsulated drug may be added to the gelling compound in the mixing vessel, at step 114 (FIG. 1) of the manufacturing process, and the gummy candies are tested once again. If the gummy candies meet the label requirements, then the chemical formulation will be set (with an encapsulated drug), and the gummies may be mass produced and packaged using the second method of incorporation described above.

If the encapsulated gummy cadies do not meet the label requirements, then the drug may need to be incorporated into the cooked candy as an oil, extract, or liquid in the flavoring and coloring phase of the manufacturing process (step 240). In this step, a fourth test batch may be produced where a liquid or extract drug may be added to the cooked candy with the coloring and flavoring at step 122 of the manufacturing process. After the gummy candies are produced, the batch may be tested once again to validate the drug composition of the candies. If the drug composition is validated, then the chemical formulation of the gummy product is set and the gummy candies may be mass produced and packaged using the third method of incorporation described above. If the third batch does not meet the label requirements, the dosage of the liquid or extract may need to be adjusted accordingly at step 122 (FIG. 1).

The process described above may only apply to drugs generally sold in granule, solid, or powder form. Any drugs generally sold in oil, liquid, or extract form may be automatically added to the cooked candy in the flavoring and coloring phase of the manufacturing process.

Delivery systems of the present invention not only make drugs palatable, the chewy consistency of the delivery system allows drugs to be easily digested by users of all ages, particularly, those users who have problems swallowing pills. In addition, the sugar formulation of the delivery system enhances the absorption of drugs into the blood stream. Also, for users who cannot digest large drug dosages, the chewable drugs of the present invention will allow these users to administer smaller drug dosages at one time (i.e., the user can take five 10 mg gummies instead of taking one 50 mg drug dosage), which will allow the body to quickly absorb the drug.

### EXAMPLES

The following examples describe particular formulations and concentrations thereof for preparing chewable drugs of the present invention. Chewable drugs of the present invention may include non-organic and/or organic compositions. For example, in one implementation, the chewable drug may include a non-organic or an organic gummy candy. While the process of manufacturing a non-organic gummy and an organic gummy are similar, as described above, the formulations for the two systems differ, as explained in more detail below.

### Non-Organic Drug

In one implementation, the delivery system of the present invention may include a non-organic gummy. For example, a 50 mg non-organic chewable aspirin in accordance with the present invention may be prepared using the following formula:

TABLE B
NON-ORGANIC GUMMY FORMULA

| Ingredients | Content (by Weight) |
|---|---|
| Lactic acid | 1% |
| Citric Acid | 1% |
| Sucrose | 38.3% |
| Corn Syrup | 50.0% |
| Gelatin | 7% |
| Aspirin (50 mg) | 0.2% |
| Flavoring (natural/artificial) | 1.5% |
| Colorant (natural/artificial) | 1.0% |

In this example, about 50 lbs of warm water may be mixed with about 50 lbs of gelatin in the mixing tank, to form 100 lbs of gelling compound having a homogeneous 50/50 blend of water and gelatin. While gelatin is described as the binding agent in this specific example, pectin, food starch, gum, or any combination thereof may be used as the binding agent in other implementations. About 0.1% to 10% of sodium bisulfate by weight may be added to the gelling compound to reduce the pH of the gelling compound to about 3.5.

In the mixing weigh vessel, the gelling compound may be mixed with about 6 lbs of water, 38.3 lbs of sucrose, and 50 lbs of corn syrup to form the candy slurry. Because aspirin is not a heat sensitive drug, about 0.15 lbs to 0.2 lbs of aspirin may be added to the candy slurry at step 114 (FIG. 1). About 0.1% sodium citrate by weight may also be added to the candy slurry to maintain the pH of the slurry at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum chamber, where the candy may be further dehydrated to a brix of about 80. After leaving the vacuum, the cooked candy is placed in the dosier where about 1.5% of strawberry flavoring by weight and about 1% of red cabbage coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### Organic Drug

In another implementation, the delivery system of the present invention may include an organic gummy. To create an organic gummy, the ingredients used to form the drug must meet the requirements for organic certification. These ingredients may include, but not be limited to, pectin, organic evaporated cane juice, organic tapioca syrup, organic grape juice, citric acid, lactic acid, sodium citrate, natural color (e.g., black carrot, juice concentrate, annatto, turmeric, purple berry concentrate) and natural flavor (e.g., strawberry, orange, pineapple, grape), and a proprietary blend of vitamins, minerals and other functional ingredients.

For example, an organic chewable drug containing 2 mg of Valium®, in accordance with the present invention, may be prepared using the following formula:

TABLE C
ORGANIC GUMMY FORMULA

| Ingredients | Content (by Weight) |
|---|---|
| Lactic acid | 1% |
| Citric Acid | 1% |
| Organic sugar | 43.49% |
| Organic syrup | 50% |
| Pectin | 2% |
| Valium (diazepam) (2 mg) | 0.006-0.01% |
| Natural flavoring | 1.5% |
| Natural colorant | 1.0% |

In this example, 98 lbs of warm water may be mixed with 2 lbs of pectin in the mixing tank, to form 100 lbs of gelling compound having a homogeneous 98/2 blend of water and pectin. While pectin is described as the binding agent in this specific example, gelatin and a combination of pectin and food starch may also used as the binding agent in other implementations. About 0.1% to 10% sodium bisulfate by weight may be added to the gelling compound to reduce the pH of the gelling compound to about 3.5.

In the mixing weigh vessel, the gelling compound may be mixed with 6 lbs of water, 43.49 lbs of organic sugar, and 50 lbs of organic tapioca syrup to form the candy slurry. Because Valium® is not a heat sensitive drug, about 0.08 lbs to 0.1 lbs of Valium® may be added to the candy slurry at step 114 (FIG. 1). About 0.1% sodium citrate by weight may also be added to the candy slurry to maintain the pH of the slurry at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to about 245° F., dehydrating the slurry to a brix of about 78.

After the candy is cooked, the cooked candy is sent to the vacuum chamber, where the candy may be further dehydrated to a brix of about 80. After leaving the vacuum, the cooked candy is placed in the dosier where about 1.5% natural apple and cherry flavoring by weight and 1% tumeric and black hair juice coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 0.08% to 0.1% sugar by weight may be added to coat the candies.

After the candies are coated, they may be inspected to validate that the finished product meets the label requirements, and then packaged.

Unlike traditional non-organic gummy candies, organic gummies having a pectin base produce a gummy candy that is both elastic and has a slightly brittle gel texture with a brilliant fracture. Due to the differing properties between pectin and gelatin, different challenges are present during the manufacturing of pectin-based gummy candies. However, due to the properties of organic gummy candy, drugs provided in a pectin-based delivery system may be more easily and quickly digested over non-organic gummies, resulting in a more desirable drug delivery system.

### Starch-Based Drug

In another implementation, the delivery system of the present invention may include a pure starch-based gummy. For example, a starch-based chewable drug containing 5 mg of Prilosec OTC®, in accordance with the present invention, may be prepared using the following formula:

TABLE D
STARCH-BASED GUMMY FORMULA

| Ingredients | Content (by Weight) |
|---|---|
| Lactic acid | 1% |
| Citric Acid | 1% |
| Sucrose | 37.46% |
| Corn Syrup | 49.0% |
| Starch | 9% |
| Prilosec OTC ® (5 mg) | 0.02%-0.04% |
| Flavoring (natural/artificial) | 1.5% |
| Colorant (natural/artificial) | 1.0% |

In this example, about 91 lbs of warm water may be mixed with about 9 lbs of starch compound in the mixing tank, to form 100 lbs of gelling compound having a homogeneous 91/9 blend of water and starch. In one implementation, the starch compound may be corn starch, rice starch, modified starches, or any other suitable starch compound. About 0.1% to 10% of sodium bisulfate by weight may be added to the gelling compound to reduce the pH of the gelling compound to about 3.5.

In the mixing weigh vessel, the gelling compound may be mixed with about 6 lbs of water, 37.46 lbs of sucrose, and 49 lbs of corn syrup to form the candy slurry. About 0.03 lbs to 0.05 lbs of Prilosec OTC® may be added to the candy slurry at step 114 (FIG. 1). About 0.1% sodium citrate by weight may also be added to the candy slurry to maintain the pH of the slurry at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum chamber, where the candy may be further dehydrated to a brix of about 80. After leaving the vacuum, the cooked candy is placed in the dosier where about 1.5% of orange and cherry flavoring by weight and about 1% of annatto and tumeric coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

Starch-based gummies provide an additional benefit over gelatin-based gummies. In particular, because gelatin liquefies when heat is applied, gelatin-based gummies frequently melt when they are exposed to high temperatures during storage and transport. But starch is more stable than gelatin in high temperatures. This is because the semi-crystalline structure of starches do not fully recover once a starch is gelatinized (i.e., becomes a gel when cooked in water) and then cooled, so the starch becomes a thickened paste. If additional heat is applied to the thickened paste, the starch will not liquefy since the starch granules swell and burst during the gelatinization process. Thus, starch-based gummies may retain their gummy shape under high temperature without melting. This is ideal for gummies that are exposed to high temperatures during storage and transport.

The examples provided above are for illustrative purposes only. Formulations for chewable drugs of the present invention may vary based on the desired dosage of the active pharmaceutical ingredients and the amount of additives, sweeteners, and coloring added to the drug composition. Thus, testing will be required to arrive at a suitable composition for each chewable drug.

While implementations of the invention have been described with reference to a gummy delivery system, the invention is not limited to this application and may be readily used for any chewable composition that includes a pectin, gelatin, or starch base. For example, implementations of the invention may also be employed in organic tablets, capsules, or solid candies. The present invention may also apply to other forms of candies such as jelly beans or caramel-based candies. Further, while the dimensions of the holding and mixing vessels are provided herein by way of example only, the actual dimensions of these vessels may vary based on the amount of gelling compound and candy slurry produced in a given time period (e.g., per day).

The foregoing description of implementations has been presented for purposes of illustration and description. It is not exhaustive and does not limit the claimed invention to the precise form disclosed. Modifications and variations are possible in light of the above description or may be acquired from practicing the invention. The claims and their equivalents define the scope of the invention.

What is claimed is:

1. A chewable composition comprising:
a binding agent;
a sweetener; and
a pharmaceutical compound.

2. The chewable composition of claim 1 where the pharmaceutical compound is an over-the-counter drug.

3. The chewable composition of claim 1 where the pharmaceutical compound is a prescription drug.

4. The chewable composition of claim 1 where the binding agent is pectin.

5. The chewable composition of claim 1 where the binding agent is gelatin.

6. The chewable composition of claim 1 where the binding agent includes gelatin and pectin.

7. The chewable composition of claim 1 where the binding agent is starch.

8. The chewable composition of claim 1 where the binding agent includes starch and pectin.

9. The chewable composition of claim 1 further comprising a supplement.

10. The chewable composition of claim 9 where the supplement is a mineral.

11. The chewable composition of claim 9 where the supplement is a vitamin.

12. The chewable composition of claim 9 where the supplement is a plant-based supplement.

13. The chewable composition of claim 9 where the supplement is an enzyme.

14. The chewable composition of claim 1 where the composition qualifies as a composition capable of being certified as organic.

15. A drug delivery system comprising:
a gummy candy that includes a binding agent and a sweetener; and
a pharmaceutical compound incorporated into the gummy candy.

16. The drug delivery system of claim 15 where the pharmaceutical compound is an over-the-counter drug.

17. The drug delivery system of claim 15 where the pharmaceutical compound is a prescription drug.

18. The drug delivery system of claim 15 where the binding agent is pectin.

19. The drug delivery system of claim 15 where the binding agent is gelatin.

20. The drug delivery system of claim 15 where the binding agent includes gelatin and pectin.

21. The drug delivery system of claim 15 where the binding agent is starch.

22. The drug delivery system of claim 15 where the binding agent includes starch and pectin.

23. The drug delivery system of claim 15 further comprising a supplement.

24. The drug delivery system of claim 23 where the supplement is a mineral.

25. The drug delivery system of claim 23 where the supplement is a vitamin.

26. The drug delivery system of claim 23 where the supplement is a plant-based supplement.

27. The drug delivery system of claim 23 where the supplement is an enzyme.

28. The drug delivery system of claim 15 where the gummy candy qualifies as a composition capable of being certified as organic.

29. A method of preparing a chewable drug, the method comprising:
mixing a binding agent with water to form a gelling compound;
adding a desired dosage of a pharmaceutical compound to the gelling compound;
adding a sweetener to the gelling compound;
cooking the gelling compound to form a cooked candy;
adding flavoring to the cooked candy; and
molding and curing the cooked candy until a chewable drug is formed.

30. The method of claim 29 further including the step of testing the chewable drug to validate that dosage of the pharmaceutical compound in the chewable drug meets a desired requirement.

31. The method of claim 29 further including the step of encapsulating the pharmaceutical compound before adding the pharmaceutical compound to the gelling compound.

32. The method of claim 29 where the pharmaceutical compound is added to the cooked candy with the flavoring.

33. The method of claim 29 where the pharmaceutical compound includes an over-the-counter drug.

34. The method of claim 29 where the pharmaceutical compound includes a prescription drug.

35. The method of claim 29 where the chewable drug is non-organic.

36. The method of claim 29 where the chewable drug qualifies as a composition capable of being certified as organic.

37. The method of claim 29 further including the step of adding a supplement to the gelling compound.

38. The method of claim 37 where supplement may include a vitamin, a mineral, an antioxidant, a plant-based supplement, an amino acid, or an enzyme.
